(19) United States
(12) Patent Application Publication
(10) Pub. No.: US 2026/0191654 A1
(43) Pub. Date: Jul. 9, 2026

(54) VERTEBRAL BODY FIXING SYSTEM

(71) Applicant: Beijing Naton Medical Technology Holdings Co., Ltd., Beijing (CN)

(72) Inventors: Haiyuan Wu, Beijing (CN); Xuedong Zhang, Beijing (CN); Xun Zhang, Beijing (CN); Hongping Duan, Beijing (CN); Xiang Dong, Beijing (CN); Shufu Xu, Beijing (CN)

(73) Assignee: Beijing Naton Medical Technology Holdings Co., Ltd., Beijing (CN)

(21) Appl. No.: 19/130,827

(22) PCT Filed: Nov. 15, 2023
(86) PCT No.: PCT/CN2023/131734

§ 371 Date: Jul. 15, 2025

(30) Foreign Application Priority Data
Nov. 16, 2022 (CN) 202211437696.5

(51) Int. Cl. A61F 2/44; A61B 17/70

(52) U.S. Cl. CPC A61F 2/44; A61B 17/7002

(57) ABSTRACT

Provided is a vertebral body fixing system, comprising an artificial vertebral body (1) and a vertebral body fixing assembly (2), wherein the vertebral body fixing assembly (2) comprises a fixing rod (22) and a connecting component (21). The fixing rod (22) extends in an axial direction of the artificial vertebral body (1). The connecting component (21) is detachably connected to both the artificial vertebral body (1) and the fixing rod (22), and the position of the connecting component (21) in a height direction of the artificial vertebral body (1) is adjustable. The vertebral body fixing system has a stable structure, is convenient to adjust, and achieves good surgical effects.

### CROSS-REFERENCE TO RELATED APPLICATION

This application claims priority to and benefits of Chinese Patent Application Serial No. 202211437696.5, filed on Nov. 16, 2022, the entire content of which is incorporated herein by reference.

### FIELD

The present disclosure relates to a field of medical instruments, and more particularly to a vertebral body fixing system.

### BACKGROUND

Artificial vertebral bodies are used to treat diseases such as vertebral burst fractures, kyphosis deformity, spinal tumors and other diseases. A patient's damaged or diseased vertebral body needs to be removed during surgery, and a vertebral body replacement is used for transplantation to maintain a normal spinal load and a physiological curvature after removal. When it is necessary to replace a plurality of diseased vertebral bodies of the patient, since the implanted artificial vertebral bodies are long, and not easy to be adjusted, the spinal stability tends to be poor, so that the artificial vertebral bodies may have a risk of implantation failure, thus affecting a surgical effect.

In order to minimize the above problems, an anterior or posterior nail rod fixing system is implemented additionally in conjunction with the implanted artificial vertebral body in order to improve stability in an early stage and during a period of osseointegration. In the related art, the anterior or posterior nail rod fixing system is usually connected to the artificial vertebral body by means of a threaded connection, and the threaded connection is prone to a thread dislodgement after a certain period of using, resulting in slippage or even dislodgement of the artificial vertebral body. Even though other connection means such as snap joints have emerged to solve the problem of instability of the threaded connection, a connection position of the nail rod fixing system on the artificial vertebral body still may not be adjusted according to an actual situation, resulting in that the vertebral body fixing system is unable to be applied to different surgical situations and different patients, the adaptability is relatively poor and the cost of production and manufacturing is increased.

### SUMMARY

The present disclosure aims to solve, at least to some extent, one of the technical problems in the related art.

To this end, embodiments of the present disclosure propose a vertebral body fixing system which has a stable structure, convenient adjustment, good adaptability and great surgical effects.

The vertebral body fixing system of an embodiment of the present disclosure includes an artificial vertebral body and a vertebral body fixing assembly. The vertebral body fixing assembly includes a fixing rod and a connecting component, the fixing rod extends along an axial direction of the artificial vertebral body, the connecting component is detachably connected to both the artificial vertebral body and the fixing rod, and a position of the connecting component along a height direction of the artificial vertebral body is adjustable.

In the vertebral body fixing system according to the embodiment of the present disclosure, the artificial vertebral body is configured to be supported between an upper vertebra and a lower vertebra of a human body. Since the fixing rod extends along the axial direction of the artificial vertebral body and the position of the connecting component along the height direction of the artificial vertebral body is adjustable, the artificial vertebral body and the vertebrae of the human body are connected in series by the fixing rod and the connecting component, so as to minimize a problem of sliding of the artificial vertebral body relative to the vertebrae of the human body. In addition, the vertebral body fixing system of the embodiment of the present disclosure allows to adjust a relative position of the connecting component according to actual conditions of different patients, so that the vertebral body fixing system after implantation becomes more stable, is convenient to adjust and has a broad application scope.

In some embodiments, the artificial vertebral body includes a fixing portion, the fixing portion extends along the axial direction of the artificial vertebral body, the connecting component is detachably connected to the fixing portion and a position of the connecting component at the fixing portion is adjustable.

In some embodiments, the fixing portion is a slide rod or a slide groove.

In some embodiments, the fixing portion is a slide rod arranged on an outer side of the artificial vertebral body, the connecting component includes a clamping member and a connecting member, the clamping member is clamped to an outer side of the slide rod and a position of the clamping member along a length direction of the slide rod is adjustable, one end of the connecting member is detachably connected to the clamping member and the other end of the connecting member is detachably connected to the fixing rod.

In some embodiments, the clamping member includes a clamping body, a first clamping portion and a second clamping portion, the first clamping portion and the second clamping portion both are connected to the clamping body, the first clamping portion and the second clamping portion are spaced apart and define a clamping groove therebetween, the slide rod is clamped in the clamping groove, and the clamping body is detachably connected to the connecting member.

In some embodiments, a spacing groove is defined between the first clamping portion and the second clamping portion, one end of the spacing groove is in communication with the clamping groove, the other end of the spacing groove extends to the clamping body, and the connecting member is fitted over the clamping body so that the first clamping portion and the second clamping portion approach each other to clamp the slide rod in the clamping groove.

In some embodiments, the connecting member has a thread hole and the clamping body is threadedly fitted in the thread hole; or, the connecting member has a through hole and the clamping body passes through the through hole.

In some embodiments, the clamping body has external threads on an outer peripheral face of the clamping body, the external threads include a first thread portion and a second thread portion, the first thread portion is adjacent to the clamping groove, and the second thread portion includes at least three turns of threads; and a minor diameter of the first thread portion is greater than a minor diameter of the second thread portion, or a major diameter of the first thread portion is greater than a major diameter of the second thread portion.

In some embodiments, the connecting member includes a connecting rod, an end portion and a locking member, one end of the connecting rod is detachably connected to the clamping member, the other end of the connecting rod is connected to the end portion, the end portion has a U-shaped groove, the fixing rod passes through the U-shaped groove, and the locking member is threadedly fitted in with the U-shaped groove and abuts against the fixing rod.

In some embodiments, the other end of the connecting rod has a ball head, and the ball head is rotatably arranged at a bottom of the U-shaped groove.

In some embodiments, the artificial vertebral body includes a first support member and a second support member, the first support member is arranged at a lower end of the second support member, a position of the second support member relative to the first support member along an axial direction of the first support member is adjustable, and the connecting component is detachably connected to the first support member.

In some embodiments, the first support member includes a support body, the support body has a cavity, at least a part of the second support member is arranged with the cavity and a position of the second support member along an axial direction of the cavity is adjustable.

In some embodiments, the artificial vertebral body further includes a rotating member, the rotating member is arranged in the cavity, an outer peripheral wall of the rotating member is fitted with an inner wall of the cavity, the rotating member is rotatable only clockwise or counterclockwise around the axial direction of the cavity, the second support member coaxially passes through the rotating member and is threadedly fitted with the rotating member, and the rotating member is rotatable relative to the cavity to drive the second support member to move along an axial direction of the rotating member.

In some embodiments, the first support member further includes a first endplate, the first endplate is arranged at a lower end of the support body, the first endplate has a spike structure at a lower end face of the first endplate, and the first endplate abuts against a lower side vertebra of the artificial vertebral body.

In some embodiments, the second support member includes a second endplate and a stud, the stud passes through the rotating member and is threadedly fitted with the rotating member, the second endplate is arranged at an upper end of the stud, and the second endplate has a spike structure at an upper end face of the second endplate.

In some embodiments, the first support member includes a support body and a first mating portion connected to each other, the outer peripheral wall of the rotating member has a second mating portion, the first mating portion is configured to elastically abut against the second mating portion to allow the rotating member to be rotatable only clockwise or counterclockwise along a circumferential direction of the first support member.

In some embodiments, a lower end of the rotating member has a plurality of operation teeth extending along a circumferential direction of the rotating member, the plurality of operation teeth are uniformly spaced apart along the circumferential direction of the rotating member, the support body has an operation hole passing through the cavity, and the operation hole is arranged opposite to the operation teeth in an inner-outer direction.

In some embodiments, an upper end of the support body has a notch, the first mating portion is arranged in the notch, the first mating portion extends in a circumferential direction of the support body, and an end of the first mating portion in an extension direction of the first mating portion is connected to the support body.

In some embodiments, the second mating portion includes a plurality of ratchets, the plurality of ratchets are spaced apart along an outer peripheral face of the rotating member, the first mating portion has a protrusion on an inner side of the first mating portion, and the protrusion is configured to be arranged between two adjacent ratchets.

In some embodiments, the artificial vertebral body further includes a drive member, the drive member is connected to the first mating portion, and the drive member is configured to drive the first mating portion to move to separate the first mating portion from the second mating portion.

### BRIEF DESCRIPTION OF THE DRAWINGS

FIG. 1 is a schematic view of a vertebral body fixing system according to an embodiment of the present disclosure.

FIG. 2 is a schematic view of a clamping member and a slide rod in a vertebral body fixing system according to an embodiment of the present disclosure, before the slide rod is clamped in the clamping member.

FIG. 3 is a schematic view of a connecting member in a vertebral body fixing system according to an embodiment of the present disclosure.

FIG. 4 is an exploded view of an artificial vertebral body (a slide rod is removed) in a vertebral body fixing system according to an embodiment of the present disclosure.

FIG. 5 is a top view of a first support member (a slide rod is removed) in a vertebral body fixing system according to an embodiment of the present disclosure.

FIG. 6 is a schematic view of a clamping body in a vertebral body fixing system according to an embodiment of the present disclosure.

FIG. 7 is another schematic view of a clamping body in a vertebral body fixing system according to an embodiment of the present disclosure.

### REFERENCE SIGNS

1 artificial vertebral body,
11 first support member, 111 support body, 1111 notch, 1112 operation hole, 112 slide rod, 113 cavity, 114 first endplate, 115 first mating portion, 1151 hole, 1152 protrusion, 116 limiting member;
12 second support member, 121 stud, 122 second endplate;
13 rotating member, 131 second mating portion, 1311 ratchet, 132 operation tooth;
2 vertebral body fixing assembly;
21 connecting component, 211 clamping member, 2111 first clamping portion, 2112 second clamping portion, 2113 clamping body, 2114 clamping groove, 2115 spacing groove, 2116 first thread portion, 2117 second thread portion, 212 connecting member, 2121 connecting rod, 2122, end portion, 21221 U-shaped groove;
22 fixing rod.

### DETAILED DESCRIPTION

Embodiments of the present disclosure are described in detail below, and examples of the embodiments are shown in the accompanying drawings. The embodiments described below by reference to the accompanying drawings are illustrative and are intended to be used to explain the present disclosure and are not to be construed as limiting the present disclosure.

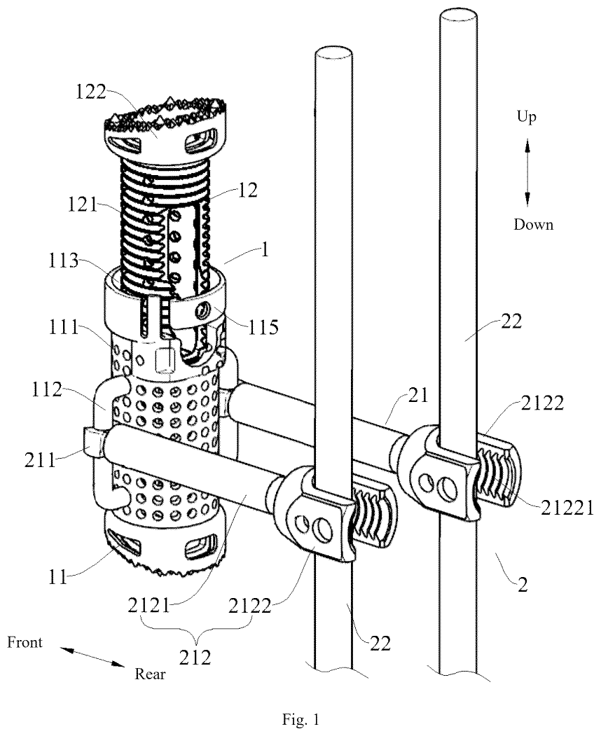

A vertebral body fixing system of an embodiment of the present disclosure is described below with reference to FIG. 1 to FIG. 7.

As shown in FIG. 1 to FIG. 5, the vertebral body fixing system of the embodiment of the present disclosure includes an artificial vertebral body 1 and a vertebral body fixing assembly 2. The vertebral body fixing assembly 2 includes a fixing rod 22 and a connecting component 21. The fixing rod 22 extends along an axial direction of the artificial vertebral body 1, the connecting component 21 is detachably connected to both the artificial vertebral body 1 and the fixing rod 22, and a position of the connecting component 21 along a height direction (e.g., an up-down direction in FIG. 1) of the artificial vertebral body 1 is adjustable.

In the vertebral body fixing system according to the embodiment of the present disclosure, the artificial vertebral body 1 is configured to be supported between an upper vertebra and a lower vertebra of a human body. Since the fixing rod 22 extends along the axial direction of the artificial vertebral body 1 and the position of the connecting component 21 along the height direction of the artificial vertebral body 1 is adjustable, the artificial vertebral body 1 and the vertebrae of the human body may be connected in series by the fixing rod 22 and the connecting component 21, so as to reduce a problem of sliding of the artificial vertebral body relative to the vertebrae of the human body. In addition, the vertebral body fixing system of the embodiment of the present disclosure allows to adjust a relative position of the connecting component 21 according to actual conditions of different patients, so that the vertebral body fixing system after implantation becomes more stable, is convenient to adjust and has a broad application scope. Therefore, the vertebral body fixing system of the embodiment of the present disclosure has advantages of a stable structure, convenient adjustment, good adaptability and great surgical results.

In some embodiments, the artificial vertebral body 1 includes a first support member 11 and a second support member 12, the first support member 11 is arranged at a lower end of the second support member 12, a position of the second support member 12 relative to the first support member 11 along an axial direction of the first support member 11 is adjustable, and the connecting component 21 is detachably connected to the first support member 11. The fixing rod 22 extends along the axial direction of the first support member 11, a plurality of connecting components 21 are provided, the plurality of connecting components 21 are arranged at intervals along a length direction of the fixing rod 22, a part of the plurality of connecting components 21 are connected to the first support member 11, and another part of the plurality of connecting components 21 may be directly connected to the vertebrae of the human body.

In the vertebral body fixing system according to the embodiment of the present disclosure, since the position of the second support member 12 relative to the first support member 11 along the axial direction of the first support member 11 is adjustable, a doctor may adjust a height of the artificial vertebral body 1 according to a length of the resected diseased vertebral body of a patient, so that the stability of the connection of the artificial vertebral body 1 and the vertebrae of the human body may be improved. Moreover, since a part of the plurality of connecting components 21 are connected to the first support member 11 and another part of the plurality of connecting components 21 are connected to the vertebrae of the human body, the artificial vertebral body 1 and the vertebrae of the human body may be connected in series by the fixing rod 22, so as to reduce the problem of sliding of the artificial vertebral body 1 relative to the vertebrae of the human body, thus further improving the stability of the vertebral body fixing system after implantation, and improving a fusion rate of the vertebral body fixing system.

In some embodiments, as shown in FIG. 1, a plurality of vertebral body fixing assemblies 2 may be provided. For example, in an embodiment of the present disclosure, two vertebral body fixing assemblies 2 are provided, and the two vertebral body fixing assemblies 2 are spaced apart along a circumferential direction of the first support member 11. The two vertebral body fixing assemblies 2 may adopt posterior fixation, and two fixing rods 22 are arranged at both sides of vertebral spinous processes along the circumferential direction of the first support member 11. The vertebral body fixing system in the embodiment of the present disclosure may further improve a fixing effect of the vertebral body fixing system and increase the fusion rate of the vertebral body fixing system by providing the two vertebral body fixing assemblies 2.

In some embodiments, as shown in FIG. 1 to FIG. 3, the connecting component 21 is detachably connected to the first support member 11 and the fixing rod 22, and a position of the connecting component 21 along a height direction of the first support member 11 is adjustable. It is to be understood that a front end of the connecting component 21 is detachably connected to the first support member 11, a rear end of the connecting component 21 is detachably connected to the fixing rod 22, and a position of the connecting component 21 along a length direction (the up-down direction as shown in FIG. 1) of the fixing rod 22 is adjustable, so that the doctor may adjust a fixation position of the connecting component 21 according to the height of the artificial vertebral body 1, and thus a connection structure of the vertebral body fixing system and the vertebrae of the human body is more reasonable and more stable.

In some embodiments, the artificial vertebral body 1 has a fixing portion, as shown in FIG. 1. In an embodiment of the present disclosure, the fixing portion is arranged on the first support member 11. The fixing portion extends along the axial direction of the artificial vertebral body 1, and the connecting component 21 is detachably connected to the fixing portion and a position of the connecting component 21 at the fixing portion is adjustable. In some embodiments, two fixing portions are provided, and the two fixing portions are spaced apart along the circumferential direction of the first support member 11, e.g., the two fixing portions are arranged at two sides of the first support member 11 in a diametral direction of the first support member 11 respectively, thereby further improving the stability of the connection of the artificial vertebral body 1 and the vertebral body fixing assembly 2.

In some embodiments, as shown in FIG. 1, the fixing portion is a slide rod 112 or a slide groove. For example, in an embodiment of the present disclosure, the fixing portion is a slide rod 112, and a length direction of the slide rod 112 is consistent with the height direction of the artificial vertebral body 1.

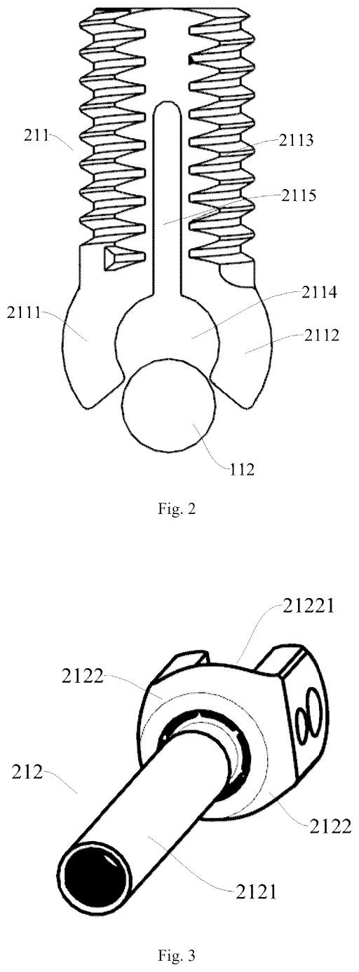

In some embodiments, as shown in FIG. 1 and FIG. 2, the first support member 11 includes a support body 111, the support body 111 has a cavity 113, and at least part of the second support member 12 is arranged in the cavity 113 and a position of the second support member 12 along an axial direction of the cavity 113 is adjustable. The slide rod 112 is arranged at an outer side of the support body 111, and both upper and lower ends of the slide rod 112 may be connected to the support body 111, so as to improve the stability of the connection of the slide rod 112 and the support body 111.

As shown in FIG. 1 to FIG. 3, the connecting component 21 includes a clamping member 211 and a connecting member 212, the clamping member 211 is clamped to an outer side of the slide rod 112 and a position of the clamping member 211 along the length direction of the slide rod 112 is adjustable. One end of the connecting member 212 is detachably connected to the clamping member 211, and the other end of the connecting member 212 is detachably connected to the fixing rod 22 and a position of the other end of the connecting member 212 along the length direction of the fixing rod 22 is adjustable. It is to be understood that the clamping member 211 may clamp to the slide rod 112 at different positions of the slide rod 112 along a height direction of the slide rod 112, thereby adjusting a position of the connecting component 21 in the up-down direction. Since any two of the slide rod 112, the clamping member 211, the connecting member 212, and the fixing rod 22 are detachably connected to each other, each part may be processed and manufactured separately and then be assembled. Therefore, the connecting component 21 of the vertebral body fixing system in the embodiment of the present disclosure has a reasonable structural design, convenient manufacturing and processing, and great use effects.

In some embodiments, as shown in FIG. 2 and FIG. 3, the clamping member 211 includes a clamping body 2113, a first clamping portion 2111 and a second clamping portion 2112, the first clamping portion 2111 and the second clamping portion 2112 are both connected to the clamping body 2113, and the first clamping portion 2111 and the second clamping portion 2112 are elastic. The first clamping portion 2111 and the second clamping portion 2112 are spaced apart and define a clamping groove 2114, the slide rod 112 is clamped in the clamping groove 2114, and the clamping body 2113 is detachably connected to the connecting member 212. For example, the clamping member 211 is a one-piece member, thereby facilitating the processing and manufacturing of the clamping member 211. The first clamping portion 2111 and the second clamping portion 2112 are arranged symmetrically along a diametral direction of the slide rod 112. The first clamping portion 2111 and the second clamping portion 2112 have an elastic force for clamping the slide rod 112 to avoid disengagement of the slide rod 112 from the clamping groove 2114. In the vertebral body fixing system of the embodiment of the present disclosure, the clamping member 211 is configured to have the above structure, so that the clamping member 211 is simple in structure, and easy to be processed and manufactured, and also facilitates adjusting the position of the clamping member 211 along the length direction of the slide rod 112, thus providing high maneuverability.

In other embodiments, when the fixing portion is the slide groove, the slide groove may be formed in the support body 111 along an axial direction of the support body 111. In this case, the clamping member 211 may be a bump, and the bump is slidably arranged in the slide groove, so as to adjust the position of the connecting component 21 at the artificial vertebral body 1 by a sliding fit of the bump and the slide groove.

In some embodiments, as shown in FIG. 2 and FIG. 3, an outer peripheral outline of the clamping groove 2114 between the first clamping portion 2111 and the second clamping portion 2112 has an arc shape with an opening, and an outer peripheral outline of the slide rod 112 is cylindrical, thereby ensuring that the slide rod 112 may not only be clamped in the clamping groove 2114, but also may rotate relative to the clamping groove 2114, i.e., not only a position of the clamping member 211 along an axial direction on the slide rod 112 is adjustable, but also the clamping member 211 is rotatable around the slide rod 112 to adjust an angle of the entire vertebral body fixing assembly, so as to adapt to different surgical situations, thus further improving the adaptability of the vertebral body fixing system. A spacing groove 2115 is arranged between the first clamping portion 2111 and the second clamping portion 2112, one end of the spacing groove 2115 is in communication with the clamping groove 2114, and the other end of the spacing groove 2115 extends to the clamping body 2113. The connecting member 212 is fitted over the clamping body 2113 so that the first clamping portion 2111 and the second clamping portion 2112 approach each other to clamp the slide rod 112 in the clamping groove 2114. It is to be understood that the spacing groove 2115 is arranged to enable the first clamping portion 2111 and the second clamping portion 2112 to move to a certain extent, so that the first clamping portion 2111 and the second clamping portion 2112 may move away from each other to make the opening of the clamping groove 2114 larger in order to accommodate the slide rod 112, and also, under an action of the connecting member 212, the first clamping portion 2111 and the second clamping portion 2112 may move close to each other to make the opening of the clamping groove 2114 smaller in order to avoid disengagement of the slide rod 112 from the clamping groove 2114. A length of the spacing groove 2115 is not specifically limited, as long as sufficient deformation of the first clamping portion 2111 and the second clamping portion 2112 is ensured.

In some embodiments, the connecting member 212 has a thread hole, and the clamping body 2113 is fitted in the thread hole by threads, so that the clamping groove 2114 has an elastic force for clamping the slide rod 112. Alternatively, the connection member 212 has a through hole, and the clamping body 2113 passes through the through hole and has an interference fit with the through hole.

For example, as shown in FIG. 2, an outer diameter of the slide rod 112 is substantially the same as an inner diameter of the clamping groove 2114, and a central angle corresponding to the arc outline of the clamping groove 2114 is greater than 180 degrees. When the clamping member 211 needs to be clamped to the slide rod 112, the clamping body 2113 may first be separated from the connecting member 212 or the connecting member 212 may avoid the spacing groove 2115. Due to the function of the spacing groove 2115, the first clamping portion 2111 and the second clamping portion 2112 may be easily spread apart, so as to facilitate clamping the slide rod 112 into the clamping groove 2114. After the slide rod 112 is clamped, the clamping body 2113 may be threadedly fitted in the thread hole of the connecting member 212 and the connecting member 212 may be screwed so that the connecting member 212 moves close to the clamping groove 2114, and at this time, the spacing groove 2115 is located in the thread hole, thereby preventing the first clamping portion 2111 and the second clamping portion 2112 from being spread apart, so as to avoid disengagement of the slide rod 112 from the clamping groove 2114. In the vertebral body fixing system of the embodiment of the present disclosure, by providing the clamping member 211 with the above structure, the clamping member 211 and the slide rod 112 may be connected reliably, be simple in structure and be assembled conveniently, and have great use effects.

As another example, as shown in FIG. 2, the outer diameter of the slide rod 112 is slightly larger than the inner diameter of the clamping groove 2114, so that when the slide rod 112 is clamped in the clamping groove 2114, the first clamping portion 2111 and the second clamping portion 2112 are elastically deformed so as to expand threads of an end of the clamping body 2113 adjacent to the first clamping portion 2111 (the second clamping portion 2112). When the clamping body 2113 is fitted in the thread hole of the connecting member 212, a front end of the clamping body 2113 is expanded so that the front end of the clamping body 2113 is in interference fit with the thread hole of the connecting member 212, thus resulting in a more secure connection of the clamping member 211 and the connecting member 212. In other embodiments, the connecting member 212 may directly have a through hole, and the clamping body 2113 passes through the through hole and is in interference fit with the through hole, which also enables the first clamping portion 2111 and the second clamping portion 2112 to move close to each other for tightening, so as to avoid disengagement of the slide rod 112.

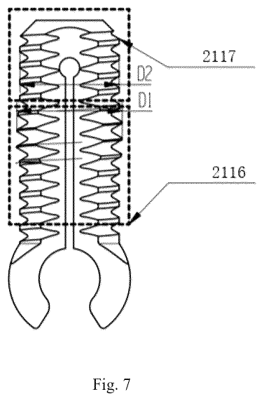

In some embodiments, as shown in FIG. 6-FIG. 7, the clamping body 2113 has an external thread on an outer peripheral face of the clamping body 2113, the external thread includes a first thread portion 2116 and a second thread portion 2117, the first thread portion 2116 is adjacent to the clamping groove 2114, and the second thread portion 2117 includes at least three turns of threads. A minor diameter d1 of the first thread portion 2116 is larger than a minor diameter d2 of the second thread portion 2117, or, a major diameter D1 of the first thread portion 2116 is larger than a major diameter D2 of the second thread portion 2117. It is understood that when the clamping body 2113 is connected in the thread hole of the connecting member 212, the second thread portion 2117 firstly enters the thread hole of the connecting member 212, and with further screwing, when the entire second thread portion 2117 enters the thread hole and the first thread portion 2116 is to be connected to threads of the thread hole, since the minor diameter of the first thread portion 2116 is larger than the minor diameter of the second thread portion 2117 or the major diameter of the first thread portion 2116 is larger than the major diameter of the second thread portion 2117, an engagement of the first thread portion 2116 with the thread hole will be a little bit more difficult than an engagement of the second thread portion 2117 with the thread hole, which makes the first clamping portion 2111 and the second clamping portion 2112 move much closer to each other to further clamp the slide rod 112. Moreover, the fit of the first thread portion 2116 with the thread hole also has a certain damping effect, and an operator may be reminded that a screwing position of the clamping body 2113 has reached the first thread portion 2116. Since the second thread portion 2117 has at least three turns of threads, it may be ensured that the connection of the second thread portion 2117 and the threaded hole is reliable. Further, the operator may also be reminded that the connection of the clamping body 2113 and the connecting member 212 is finished.

In some embodiments, as shown in FIG. 1 and FIG. 3, the connecting member 212 includes a connecting rod 2121, an end portion 2122, and a locking member (not shown), one end of the connecting rod 2121 is detachably connected to the clamping member 211, the other end of the connecting rod 2121 is connected to the end portion 2122, the end portion 2122 has a U-shaped groove 21221, the fixing rod 22 passes through the U-shaped groove 21221, the locking member is fitted with the U-shaped groove 21221 by threads, and the locking member abuts against the fixing rod 22. It is to be understood that the fixing rod 22 is arranged in the U-shaped groove 21221 and is locked by the locking member, thus facilitating detachment and installation of the fixing rod 22, and a connection position of the connecting member 212 and the fixing rod 22 may be adjusted to improve the adaptability of the vertebral body fixing system.

For example, the other end of the connecting rod 2121 has a ball head (not shown), and the ball head is rotatably arranged at a bottom of the U-shaped groove. It is to be understood that the other end of the connecting rod 2121 and the end portion 2122 are fitted with each other by means of a universal ball head, so that the end portion 2122 may be rotated relative to the connecting rod 2121, which in turn facilitates adjusting the U-shaped groove 21221 of the end portion 2122 to an arbitrary angle so as to allow the fixing rod 22 to pass through the U-shaped groove 21221 conveniently.

For example, the locking member may be a screw plug, an inner wall of the U-shaped groove 21221 has internal threads, and the locking member is fitted with the inner wall of the U-shaped groove 21221 by threads so that the locking member may abut against the fixing rod 22. The vertebral body fixing system of the embodiment of the present disclosure may facilitate fixation of the fixing rod 22, and provide easy assembling and high maneuverability, by providing the connecting member 212 with the above structure.

It is to be understood that an end of the connecting component 21 facing away from the fixing rod 22 and connected to the vertebrae of the human body may be a screw structure, and an end of the connecting component 21 facing away from the fixing rod 22 and connected to the artificial vertebral body 1 may be a clamping structure as described in the above embodiment. The vertebral body fixing system of the embodiment of the present disclosure may facilitate fixation of the artificial vertebral body 1 and the vertebrae of the human body by providing the connecting component 21 with the above structure, so that the vertebral body fixing system has a more secure connection and a higher stability.

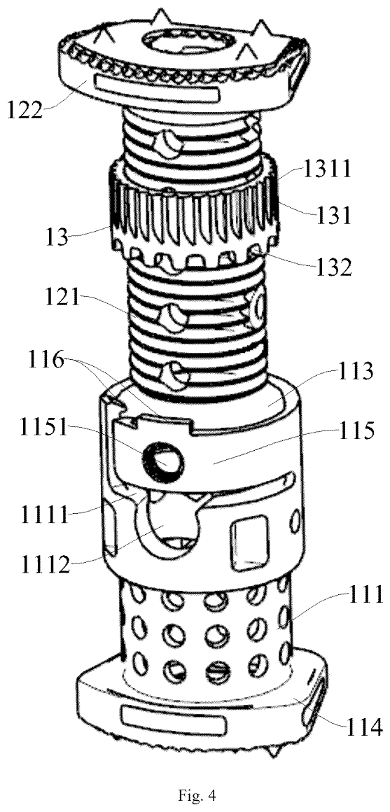

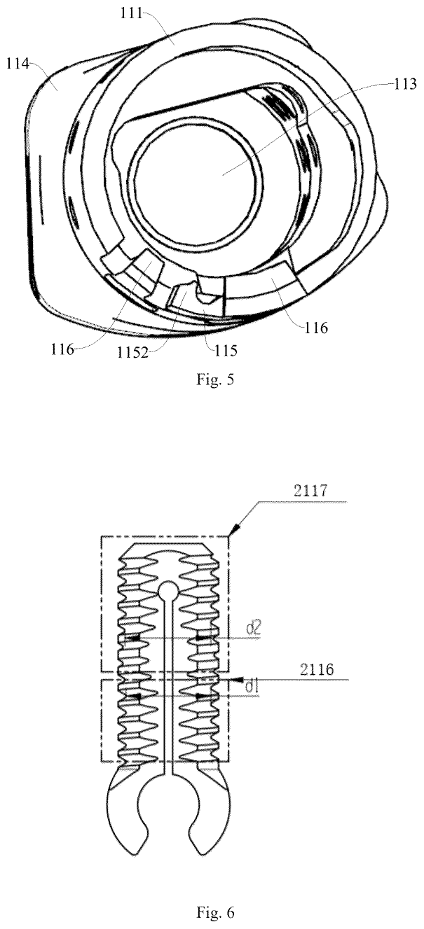

In some embodiments, as shown in FIG. 1, FIG. 4 and FIG. 5, the first support member 11 has the cavity 113, the artificial vertebral body 1 further includes a rotating member 13, the rotating member 13 is arranged in the cavity 113, an outer peripheral wall of the rotating member 13 is fitted with an inner wall of the cavity 113, and the rotating member 13 may rotate only clockwise or counterclockwise around an axial direction of the cavity 113. The second support member 12 coaxially passes through the rotating member 13 and is fitted with the rotating member 13 by threads, and the rotating member 13 is rotatable relative to the cavity 113 to drive the second support member 12 to move along an axial direction of the rotating member 13. It is to be understood that the rotating member 13 is only capable of rotating clockwise around the axial direction of the cavity 113 but not counterclockwise, or the rotating member 13 is only capable of rotating counterclockwise around the axial direction of the cavity 113 but not clockwise.

In the vertebral body fixing system according to the embodiment of the present disclosure, when the artificial vertebral body 1 needs to be expanded, the rotating member 13 may be rotated to drive the second support member 12 to move along the axial direction of the rotating member 13, and since the rotating member 13 may only rotate clockwise or counterclockwise around the axial direction of the cavity 113, the artificial vertebral body 1 may be self-locked to avoid the artificial vertebral body sinking, thus resulting in great structural stability of the artificial vertebral body 1.

It is to be understood that, as shown in FIG. 1, FIG. 4 and FIG. 5, a lower end of the second support member 12 is arranged in the cavity 113, an outer peripheral wall of the second support member 12 has an external thread, and an inner peripheral wall of the rotating member 13 has an internal thread. Thus, the second support member 12 may be driven to move upwards by unidirectional rotation of the rotating member 13 along the circumferential direction of the cavity 113. When the artificial vertebral body 1 is expanded, the expanded artificial vertebral body 1 may realize self-lock because the rotating member 13 cannot rotate reversely, thus avoiding the risk of the artificial vertebral body 1 sinking or retreating, and allowing a height of the second support member 12 to be stably maintained.

In some embodiments, as shown in FIG. 1, FIG. 4, and FIG. 5, the first support member 11 further includes a first endplate 114, the first endplate 114 is arranged at a lower end of the support body 111, a lower end face of the first endplate 114 has a spike structure, and the first endplate 114 abuts against a lower side vertebra of the artificial vertebral body 1, in order to improve the stability of the connection of the artificial vertebral body 1 and the vertebrae.

In some embodiments, as shown in FIG. 1 and FIG. 4, the second support member 12 includes a second endplate 122 and a stud 121, the stud 121 passes through the rotating member 13 and is fitted with the rotating member 13 by threads, the second endplate 122 is arranged at an upper end of the stud 121, and an upper end face of the second endplate 122 has a spike structure. It is to be understood that the second endplate 122 abuts against an upper side vertebra of the artificial vertebral body 1 in order to improve the stability of the connection of the artificial vertebral body 1 and the vertebrae.

In some embodiments, as shown in FIG. 1, FIG. 4 and FIG. 5, the first support member 11 includes the support body 111 and a first mating portion 115 connected to each other, the cavity 113 is arranged in the support body 111, an outer peripheral wall of the rotating member 13 has a second mating portion 131, and the first mating portion 115 is configured to elastically abut against the second mating portion 131 to allow the rotating member 13 to rotate only along the circumferential direction of the first support member clockwise or counterclockwise.

It is to be understood that the stud 121 is driven to move upwards by rotating the rotating member 13 clockwise, and when the vertebral body fixing system is expanded, the first mating portion 115 and the second mating portion 131 are able to realize an self-lock of the expanding mechanism, prevent the rotating member 13 from rotating counterclockwise and realize a stable maintenance of the height of the second support member 12. Alternatively, the rotating member 13 rotates counterclockwise to drive the second support member 12 to move upwards, and when the vertebral body fixing system is expanded, the first mating portion 115 and the second mating portion 131 are able to realize the self-lock of the expanding mechanism, prevent the rotating member 13 from rotating clockwise, and realize the stable maintenance of the height of second support member 12.

The vertebral body fixing system of the embodiment of the present disclosure includes the rotating member 13, the first mating portion 115 and the second mating portion 131, and the vertebral body fixing system is expanded by rotating the rotating member 13 to drive the stud 121 to move up and down. The first mating portion 115 and the second mating portion 131 are capable of realizing the self-lock of the expanding mechanism of the vertebral body fixing system, thus realizing the stable maintenance of the height of the vertebral body fixing system. The first mating portion 115 is arranged on the inner peripheral face of the cavity 113 and the second mating portion 131 is arranged on the rotating member 13, so that there is no specific requirement for a thickness of an outer wall of the support body 111, and the space for bone implantation may not be affected due to the incorporation of the first mating portion 115 and the second mating portion 131.

As shown in FIG. 4 and FIG. 5, in an embodiment of the present disclosure, a lower end of the rotating member 13 has a plurality of operation teeth 132 extending along a circumferential direction of the rotating member 13, and the plurality of operation teeth 132 are uniformly spaced apart along the circumferential direction of the rotating member 13. The support body 111 has an operation hole 1112 passing through the cavity 113, and the operation hole 1112 is arranged opposite to the operation teeth 132 in an inner-outer direction, so that a rotating handle (not shown) may pass through the operation hole 1112 to be fitted with the operation teeth 132 of the rotating member 13, thereby driving the rotating member 13 to rotate in the support body 111 by rotating the rotating handle. It is to be understood that an end of the rotating handle connected in the operation hole 1112 includes a gear structure, and the gear structure is capable of meshing with the operation teeth 132, so that the rotating handle may be rotated to drive the rotating member 13 to rotate. The rotating handle and the gear structure are known in the related art, which are not repeated herein.

In some embodiments, as shown in FIG. 4 and FIG. 5, the first mating portion 115 has the ability of elastic deformation, and the first mating portion 115 is located at an upper end of the support body 111 and is arranged opposite to the rotating member 13 in the inner-outer direction. When a height of the stud 121 needs to be decreased, the first mating portion 115 may be pulled to move outwards in the inner-outer direction, or pulled to move upwards or downwards, so that the first mating portion 115 and the second mating portion 131 on the rotating member 13 are disengaged, thereby realizing that the first mating portion 115 and the second mating portion 131 are unlocked, so that the rotating member 13 may rotate reversely in order to lower a position of the stud 121. When the position of the stud 121 is adjusted to a suitable position, the first mating portion 115 may be released, and since the first mating portion 115 has the ability of elastic deformation, the first mating portion 115 may be automatically fitted with the second mating portion 131 to prevent the stud 121 from sinking.

In some embodiments, as shown in FIG. 4 and FIG. 5, the support body 111 has a notch 1111 at an upper end of the support body 111, the first mating portion 115 is arranged in the notch 1111, the first mating portion 115 extends in a circumferential direction of the support body 111, and one end of the first mating portion 115 in an extension direction of the first mating portion 115 is connected to the support body 111. In some embodiments, the support body 111 is a sleeve without a top face, an inner peripheral face of the sleeve defines the cavity 113, and the rotating member 13 is rotatably arranged in the cavity 113. A side face of an upper end of the support body 111 has the notch 1111 that extends through the support body 111 in the inner-outer direction. The first mating portion 115 is arranged in the notch 1111 and one side of the first mating portion 115 is connected to an inner peripheral face of the notch 1111. An upper end face of the first mating portion 115 is flush with an upper end face of the support body 111, and each of a bottom face of the first mating portion 115 and the other side of the first mating portion 115 is spaced apart from the inner peripheral face of the notch 1111. When the height of the stud 121 needs to be decreased, the first mating portion 115 may be pulled out of the notch 1111, thereby facilitating the disengagement of the first mating portion 115 and the second mating portion 131.

In the embodiment of the present disclosure, a stepped face (not shown) is arranged in the cavity 113. When the rotating member 13 is arranged in the cavity 113, the operation teeth 132 of the rotating member 13 may abuts against the stepped face, so that a position of the rotating member 13 in the cavity 113 may be limited by the stepped face. Moreover, the first mating portion 115 and the support body 111 may be integrally formed to ensure stability of the overall structure. In some embodiments, the first mating portion 115 is formed in the support body 111 by cutting the support body 111, for example, via wire cutting or the like. In other embodiments, the first mating portion 115 and the support body 111 may also be processed by other processing means, or the first mating portion 115 and the support body 111 may be configured as separate structures.

In some embodiments, as shown in FIG. 4 and FIG. 5, the second mating portion 131 includes a plurality of ratchets 1311, the plurality of ratchets 1311 are spaced apart along an outer peripheral face of the rotating member 13, the first mating portion 115 has a protrusion 1152 on an inner side of the first mating portion 115, and the protrusion 1152 may be arranged between two adjacent ratchets 1311. When the rotating member 13 is rotated clockwise or counterclockwise, the protrusion 1152 may slide across the ratchets 1311, and the protrusion 1152 may prevent the rotating member 13 from rotating reversely.

As shown in FIG. 4 and FIG. 5, it is to be understood that the plurality of ratchets 1311 may rotate synchronously with the rotating member 13, and the protrusion 1152 is located on an inner peripheral face of the first mating portion 115, an inner peripheral face of the protrusion 1152 extends in a clockwise direction and is inclined inwards in the inner-outer direction, and the protrusion 1152 may have a curved face or a flat face. When the height of the stud 121 needs to be increased, the plurality of ratchets 1311 rotate clockwise with the rotating member 13, and the protrusion 1152 may be switched into a tooth groove between any two adjacent ratchets 1311. When the rotating member 13 drives the plurality of ratchets 1311 to rotate by an angle, the ratchets 1311 may slide across the protrusion 1152, so as to enable the rotating member 13 to rotate smoothly. When the rotating member 13 rotates counterclockwise, a side face of the protrusion 1152 will be jammed against the ratchets 1311, so that the rotating member 13 is prevented from rotating counterclockwise. When the height of the stud 121 needs to be decreased, the first mating portion 115 may be pulled to drive the protrusion 1152 to disengage from the ratchets 1311, thereby allowing the rotating member 13 to rotate counterclockwise.

In some embodiments, when the height of the stud 121 needs to be increased, the plurality of ratchets 1311 rotate counterclockwise with the rotating member 13, and the protrusion 1152 may be switched into the tooth groove between any two adjacent ratchets 1311. When the rotating member 13 drives the plurality of ratchets 1311 to rotate by an angle, the ratchets 1311 may slide across the protrusion 1152, thereby allowing the rotating member 13 to rotate smoothly. When the rotating member 13 rotates clockwise, a side face of the protrusion 1152 will be jammed against the ratchets 1311, so that the rotating member 13 is prevented from rotating clockwise. When the height of the stud 121 needs to be decreased, the first mating portion 115 may pulled to drive the protrusion 1152 to disengage from the ratchets 1311, thereby allowing the rotating member 13 to rotate clockwise.

In some embodiments, as shown in FIG. 4 and FIG. 5, the artificial vertebral body further includes a drive member (not shown), the drive member is connected to the first mating portion 115, and the drive member may drive the first mating portion 115 to move, so as to disengage the first mating portion 115 from the second mating portion 131. In some embodiments, the drive member may be a pin and pass through the first mating portion 115. The first mating portion 115 may be driven by the drive member to move away from the rotating member 13, so as to separate the first mating portion 115 from the second mating portion 131.

In some embodiments, as shown in FIG. 4 and FIG. 5, the first mating portion 115 has a hole 1151 passing through the cavity 113, and the drive member is fitted in the hole 1151 by threads. For example, the drive member has an external thread, and the hole 1151 has an internal thread fitted with the external thread, so that the drive member is fixed to the first mating portion 115 by a fit of the internal thread and the external thread, thus preventing the drive member from disengaging from the first mating portion 115, and ensuring the efficiency of the drive member driving the first mating portion 115 to move.

It is to be understood that the drive member may be fixed in the hole 1151 by snap-fit, interference fit, etc., and the hole 1151 and the operation hole 1112 are arranged opposite to each other in the up-down direction. In some embodiments, the hole 1151 and the operation hole 1112 are arranged opposite each other in the up-down direction, during the vertebral implantation, a dual-channel tube may be implanted into the human body, so that two channels of the dual-channel tube are aligned with the hole 1151 and the operation hole 1112, respectively, and then a operation handle or the drive member may be extended into the dual-channel tube, which simplifies a surgical process. Moreover, the hole 1151 may be a full hole or a hole larger than a half hole. In other embodiments, the hole 1151 and the operation hole 1112 may be misaligned in the up-down direction.

In some embodiments, as shown in FIG. 4 and FIG. 5, the support body 111 and/or the first mating portion 115 has a limiting member 116, the limiting member 116 extends inwards along a radial direction toward the cavity 113, and the limiting member 116 abuts against an end of the rotating member 13 adjacent to the second support member to prevent the rotating member 13 from disengaging out of the cavity 113. For example, the limiting member 116 is a snap protrusion, the snap protrusion may be arranged at an upper edge of the support body 111 or at an upper edge of the first mating portion 115. When the rotating member 13 needs to be placed in the cavity 113, the first mating portion 115 may be pulled apart by the drive member, so that the snap protrusion will not interfere with the rotating member 13. After the rotating member 13 is arranged in place, the first mating portion 115 is released, and then the snap protrusion may play a role of limiting the rotating member 13. These operations are simple and convenient.

In the description of the present disclosure, it is to be understood that the terms such as “central”, “longitudinal”, “transverse”, “length”, “width”, “thickness”, “up”, “down”, “front”, “rear”, “left”, “right”, “vertical”, “horizontal”, “top”, “bottom”, “inner”, “outer”, “clockwise”, “counterclockwise”, “axial”, “radial”, “circumferential” etc., indicate orientations or positional relationships based on those shown in the drawings, are only intended to facilitate the description of the present disclosure and to simplify the description, and are not intended to indicate or imply that the device or element referred to must have a particular orientation, and be constructed and operated in a particular orientation. Therefore, these terms cannot be understood as a limitation of the present disclosure.

Moreover, the terms “first” and “second” are used for purposes of description only and cannot be understood as indicating or implying relative importance or implicitly specifying the number of technical features indicated. Thus, a feature defined with the term “first” or “second” may expressly or impliedly include at least one such feature. In the description of the present disclosure, “a plurality of” means at least two, such as two, three, etc., unless expressly and specifically limited otherwise.

In the present disclosure, unless expressly specified or limited otherwise, the terms “mount”, “interconnect”, “connect”, “fix”, and the like should be understood broadly and may indicate, such as a fixed connection, a detachable connection, or a one-piece unit; a mechanical connection, an electrical connection, or a communication with each other; a direct connection, an indirect connection through an intermediate media, and intercommunication or interaction of two elements, unless expressly limited otherwise. For those skilled in the art, the specific meaning of the above terms in the present disclosure may be understood according to the specific situations.

In the present disclosure, unless expressly specified or limited otherwise, a first feature being “above” or “below” a second feature may indicate a direct contact between the first feature and the second feature, or an indirect contact between the first feature and the second feature through an intermediate medium. Furthermore, the first feature being “on”, “above” and “over” the second feature may indicate that the first feature is directly above or obliquely above the second feature, or only indicate that the first feature has a height larger than the second feature. The first feature being “below”, “under” and “underneath” the second feature may indicate that the first feature is directly below or obliquely below the second feature, or only indicate that the first feature has a height less than the second feature.

In the present disclosure, the terms “an embodiment”, “some embodiments”, “an example”, “a specific example”, or “some examples” mean that the specific features, structures, materials, or characteristics described in connection with the embodiments or examples are included in at least one embodiment or example of the present disclosure. In the specification, schematic expressions of the above terms do not necessarily refer to the same embodiment or example. Furthermore, the specific features, structures, materials, or characteristics described may be combined in any one or more embodiments or examples in a suitable manner.

Although the embodiments of the present disclosure have been shown and described above, it may be understood that the above embodiments are illustrative and are not to be understood as limitations of the present disclosure, and that those skilled in the art may make changes, modifications, substitutions, and variations of the above embodiments within the scope of the present disclosure.

1. A vertebral body fixing system, comprising,
an artificial vertebral body;
a vertebral body fixing assembly, wherein the vertebral body fixing assembly comprises a fixing rod and a connecting component, the fixing rod extends along an axial direction of the artificial vertebral body, the connecting component is detachably connected to both the artificial vertebral body and the fixing rod, and a position of the connecting component along a height direction of the artificial vertebral body is adjustable.

2. The vertebral body fixing system according to claim 1, wherein the artificial vertebral body has a fixing portion, the fixing portion extends along the axial direction of the artificial vertebral body, the connecting component is detachably connected to the fixing portion and a position of the connecting component at the fixing portion is adjustable.

3. The vertebral body fixing system according to claim 2, wherein the fixing portion is a slide rod or a slide groove.

4. The vertebral body fixing system according to claim 2, wherein the fixing portion is a slide rod arranged at an outer side of the artificial vertebral body, the connecting component comprises a clamping member and a connecting member, the clamping member is clamped to an outer side of the slide rod and a position of the clamping member along a length direction of the slide rod is adjustable, one end of the connecting member is detachably connected to the clamping member, and the other end of the connecting member is detachably connected to the fixing rod.

5. The vertebral body fixing system according to claim 4, wherein the clamping member comprises a clamping body, a first clamping portion and a second clamping portion, the first clamping portion and the second clamping portion both are connected to the clamping body, the first clamping portion and the second clamping portion are spaced apart and define a clamping groove therebetween, the slide rod is clamped in the clamping groove, and the clamping body is detachably connected to the connecting member.

6. The vertebral body fixing system according to claim 5, wherein a spacing groove is defined between the first clamping portion and the second clamping portion, one end of the spacing groove is in communication with the clamping groove, the other end of the spacing groove extends to the clamping body, and the connecting member is fitted over the clamping body so that the first clamping portion and the second clamping portion approach each other to clamp the slide rod in the clamping groove.

7. The vertebral body fixing system according to claim 6, wherein the connecting member has a thread hole, and the clamping body is threadedly fitted in the thread hole; or
the connecting member has a through hole, and the clamping body passes through the through hole.

8. The vertebral body fixing system according to claim 7, wherein the clamping body has external threads on an outer peripheral face of the clamping body, the external threads comprise a first thread portion and a second thread portion, the first thread portion is adjacent to the clamping groove, and the second thread portion comprises at least three turns of threads; and
a minor diameter of the first thread portion is larger than a minor diameter of the second thread portion, or a major diameter of the first thread portion is larger than a major diameter of the second thread portion.

9. The vertebral body fixing system according to claim 4, wherein the connecting member comprises a connecting rod, an end portion and a locking member, one end of the connecting rod is detachably connected to the clamping member, the other end of the connecting rod is connected to the end portion, the end portion has a U-shaped groove, the fixing rod passes through the U-shaped groove, and the locking member is threadedly fitted in the U-shaped groove and abuts against the fixing rod.

10. The vertebral body fixing system according to claim 9, wherein the other end of the connecting rod has a ball head, and the ball head is rotatably arranged at a bottom of the U-shaped groove.

11. The vertebral body fixing system according to claim 1, wherein the artificial vertebral body comprises a first support member and a second support member, the first support member is arranged at a lower end of the second support member, a position of the second support member relative to the first support member along an axial direction of the first support member is adjustable, and the connecting component is detachably connected to the first support member.

12. The vertebral body fixing system according to claim 2, wherein the first support member comprises a support body, the support body has a cavity, at least a part of the second support member is arranged in the cavity and a position of the second support member along an axial direction of the cavity is adjustable.

13. The vertebral body fixing system according to claim 12, wherein the artificial vertebral body further comprises a rotating member, the rotating member is arranged in the cavity, an outer peripheral wall of the rotating member is fitted with an inner wall of the cavity, the rotating member is rotatable only clockwise or counterclockwise around the axial direction of the cavity, the second support member coaxially passes through the rotating member and is threadedly fitted with the rotating member, and the rotating member is rotatable relative to the cavity, to drive the second support member to move along an axial direction of the rotating member.

14. The vertebral body fixing system according to claim 13, wherein the first support member further comprises a first endplate, the first endplate is arranged at a lower end of the support body, the first endplate has a spike structure at a lower end face of the first endplate, and the first endplate abuts against a lower side vertebra of the artificial vertebral body.

15. The vertebral body fixing system according to claim 14, wherein the second support member comprises a second endplate and a stud, the stud passes through the rotating member and is threadedly fitted with the rotating member, the second endplate is arranged at an upper end of the stud, and an upper end face of the second endplate has a spike structure.

16. The vertebral body fixing system according to claim 13, wherein the first support member comprises a support body and a first mating portion connected to each other, an outer peripheral wall of the rotating member has a second mating portion, and the first mating portion is configured to elastically abut against the second mating portion to allow the rotating member to be rotatable only clockwise or counterclockwise along a circumferential direction of the first support member.

17. The vertebral body fixing system according to claim 13, wherein a lower end of the rotating member has a plurality of operation teeth extending along a circumferential direction of the rotating member, the plurality of operation teeth are uniformly spaced apart along the circumferential direction of the rotating member, the support body has an operation hole passing through the cavity, and the operation hole is arranged opposite to the operation teeth in an inner-outer direction.

18. The vertebral body fixing system according to claim 16, wherein the support body has a notch at an upper end of the support body, the first mating portion is arranged in the notch, the first mating portion extends along a circumferential direction of the support body, and an end of the first mating portion in an extension direction of the first mating portion is connected to the support body.

19. The vertebral body fixing system according to claim 16, wherein the second mating portion comprises a plurality of ratchets, the plurality of ratchets are spaced apart along an outer peripheral face of the rotating member, the first mating portion has a protrusion on an inner side of the first mating portion, and the protrusion is configured to be arranged between two adjacent ratchets.

20. The vertebral body fixing system according to claim 16, wherein the artificial vertebral body further comprises a drive member, the drive member is connected to the first mating portion, and the drive member is configured to drive the first mating portion to move to separate the first mating portion from the second mating portion.
